(19) United States
(12) Patent Application Publication
(10) Pub. No.: US 2008/0318280 A1
(43) Pub. Date: Dec. 25, 2008

(54) COVER FOR AN ARRAY OF REACTION VESSELS FOR ONE-STEP OPERATION MODUS

(75) Inventors: Arne Schafrinski, Bad Oldesloe (DE); Henner Tasch, Hamburg (DE); Lutz Timmann, Fuehlendorf (DE); Stefan Roth, Hamburg (DE); Rudiger Huhn, Luebeck (DE); Thomas Uschkureit, Hamburg (DE)

Correspondence Address: King Spalding LLP, 4 Embarcadero Center, Suite 3500, San Francisco, CA, 94111, US

(73) Assignee: Eppendorf AG, Hamburg (DE)

(21) Appl. No.: 12/030,128

(22) Filed: Feb. 12, 2008

Related U.S. Application Data
(60) Provisional application No. 60/889,616, filed on Feb. 13, 2007.

(51) Int. Cl. C12P 19/34; C12M 1/02; B23P 11/00

(52) U.S. Cl. 435/91.2; 435/303.1; 29/428

(57) ABSTRACT

The present invention relates to means for covering one or more sample(s) that are suitable to avoid or minimize evaporation and/or condensation of any vaporizable substance that may be present in the sample(s) or reaction mixture(s), in particular evaporation of substance at the fringes of a vessel or an array of vessels or condensation of said substance on the lid of a reaction vessel or a plate/block containing the sample(s) and/or the means for covering. This is achieved by providing a device comprising, among others, a means for positioning a covering means and a means for guiding at least one lid of the covering means.

### CROSS-REFERENCE TO RELATED APPLICATIONS

This application claims the benefit under 35 U.S.C. §119(e) of U.S. Provisional Application Ser. No. 60/889,616 filed Feb. 13, 2007.

### FIELD OF THE INVENTION

The present invention relates to a device and a method for performing processes and/or reactions that are conducted in a temperature-controlled environment.

### BACKGROUND OF THE INVENTION

A thermal cycler for implementing chemical and/or biological reactions comprising a body for accommodating one or more reaction vessels and a cover is disclosed, for example, in EP 1 013 342. This thermal cycler apparatus comprises a base body for accommodating one or more reaction vessels that is open at the top as well as a cover for closing off said accommodating portion. The apparatus also comprises a locking mechanism for locking the cover in a predetermined position in relation to the reaction vessels and the base body. Once the cover is closed, an electrically activated positioner is actuated so that a rigid part of said cover is moved downwards against the reaction vessel(s).

Another thermal cycler for the automatic performance of a polymerase chain reaction having a closed temperature control system is described in U.S. Pat. No. 5,575,610. In one embodiment that is disclosed in more detail, for example in FIG. 19, a structure is shown comprising a sliding cover which encloses the sample tubes. A heated platen is coupled to a screw to move up and down along a vertical axis as to press said heated platen with a defined pressure onto the reaction vessels. According to this embodiment, application of a pressure in z-direction is only performed after the sliding cover has been aligned with the reaction vessel(s).

Another device and method for thermal cycling is disclosed in U.S. Pat. No. 6,677,151. According to the teaching of U.S. '151, a sample well tray holder including the samples is first moved into the thermal cycling device by means of a simple translational movement until the sample well tray is aligned with a sample block assembly positioned beneath the sample well tray. After this, the sample block assembly is translated from this first position to a second position. In the first position, the sample block assembly permits the sample well tray to translate into alignment with the sample block assembly. Only in the second position, the sample block assembly is positioned vertically upward relative to the first position to contact the sample well tray. A similar disclosure in regard to aligning samples and sample holder with a plate and/or a cover is given in U.S. Pat. No. 6,730,883.

In view of the prior art in the field, it is an object of the present invention to provide a device and a method that allow to cover and enclose one or more sample, preferably a sample contained in a reaction vessel or in a plate or a block in a more simple and/or efficient manner than is known in the prior art. It is a further object, to provide a modus of operation for a thermal cycler that is easy and cost-effective to implement and easy to use.

### SUMMARY OF INVENTION

These and other objects are solved by a device for controlling the temperature of at least one sample, wherein the device comprises at least the following components:
at least one means for accommodating (2) at least one sample;
at least one means for heating and/or cooling (4) at least one sample;
at least one means for covering (3) at least one sample, wherein the means for covering comprise at least one lid (11, 12);
at least one means for positioning (24, 25) the means for covering (3) relative to the at least one sample;
at least one means for guiding (7) the at least one lid (11, 12) of the means for covering (3) from a position of no thermal and/or physical contact with at least one reaction vessel (1) into a position of thermal and/or physical contact with at least one sample.

The present invention also relates to a process for aligning and/or closing the cover of a device for controlling the temperature of at least one sample, wherein said process comprises at least the following steps: (i) aligning and/or closing the means for covering (3) into a position in which the device is deemed closed; (ii) bringing at least one lid (11, 12) of at least one means for covering (3) from a position of no physical and/or thermal contact with at least one sample into a position of physical and/or thermal contact with at least one reaction vessel (1).

Preferably these two process steps are performed simultaneously in one movement moving both the means for covering (3) and the lid (11, 12).

In a preferred embodiment, the means for positioning (24, 25) and the means for guiding (7) are adapted to effect a movement of the lid (11, 12) relative to the sample which within the same period of time, has both a horizontal component and a vertical component.

The lid (11, 12) preferably comprises a contact area (12) and an inclined portion (11).

In an alternative preferred embodiment, the lid comprises rolling means (14), preferably a circular belt. Therein it is preferred that both closing of the means for covering (3) and the bringing of the lid (11, 12) in physical contact within at least one sample are affected by said rolling means, preferably by the movement of a circular belt.

In accordance with the present invention, the “horizontal” plane is the plane perpendicular to the direction of gravity and commonly corresponds to the plane of the table on which the device is mounted. In normal operations, this is also the plane defined by the array of samples/reaction vessels to be covered. Correspondingly, the vertical plane runs along the direction of the gravity and is perpendicular to the plane of the table or the plane defined by an array of reaction vessels.

In a preferred embodiment, the movement having a concurrent horizontal and vertical component is effected during the process of closing the means for covering (3), in particular during the process of aligning the means for covering (3) relative to the reaction vessel (1) and/or the means for accommodating (2) so that, at the conclusion of said movement, the means for covering (3), the at least one reaction vessel (1) and the lid (11, 12) are in their final position.

The “final position” in the understanding of the present invention is the position in which the device is deemed closed and the sample(s) can be processed, preferably the reaction(s) in the reaction vessel(s) can be started.

In a further preferred embodiment, the means for positioning (24, 25) and the means for guiding (7) are adapted to align the means for covering (3) relative to the reaction vessel (1) and/or the means for accommodating (2) so that the contact area (12) of the lid is in physical and/or thermal contact with the at least one reaction vessel (1) and that the means for covering (3) and the lid (11, 12) are in their predetermined final position, i.e. position which the device is deemed closed and the sample(s) can be processed, preferably the reaction(s) in the reaction vessel(s) can be started.

The above-referenced movement is preferably one single continuous movement, further preferably a movement of the means for covering (3) relative to the means for accommodating (2) and/or the reaction vessel(s) (1) in the horizontal direction i.e. in a direction in which the means for covering (3) move parallel relative to the means for accommodating (29) and the reaction vessel(s) (1). While it is preferred that the means for covering (3) move in parallel relative to the means for accommodating (2) and/or the reaction vessel (1), during said movement, the movement of the means for guiding (7) and the contact area (12) occurring concurrently with the movement of the means for covering (3) preferably have a horizontal component and a vertical component, i.e. do not only move parallel to the means for accommodating (2) and/or the reaction vessel (1) but also, to some extent, in the vertical direction, i.e. perpendicular to the horizontal plane as defined by the means for accommodating and/or the reaction vessel(s).

The specific arrangement of means for accommodating (2), reaction vessel(s) (1), means for covering (3), means for guiding (7) and the lid (11, 12) as described above in regard to the device of the present invention allows to close and/or align the cover of the overall device in one single continuous a movement while simultaneously bringing the contact area of said lid of said means for covering (3) in physical and/or thermal contact with the at least one reaction vessel.

Among others, the closing and aligning of the cover into its final position in one continuous movement according to the present invention has the following advantages over the embodiments of the prior art: Overall, in order to close the cover and to establish tight physical and/or thermal contact between the lid (typically including means for heating and/or cooling) with the at least one reaction vessel, a comparatively simple movement is effected. This lowers or minimizes the number of mechanical components needed to perform the tasks of aligning and closing the cover and of bringing at least one part of the cover in close physical and/or thermal contact with the sample(s) and/or reaction vessel(s). By means of controlling the pressure/force as exerted by the contact area on closing the cover, different types of reaction vessels can be accommodated within the same device, in particular reaction vessels having different height and/or different height tolerances.

The present invention thus relates to a device and a method for performing processes and/or reactions that are conducted in a temperature-controlled environment. While the present invention is exemplarily discussed in the context of thermal cyclers, the device and method of the invention are not restricted to this specific application but rather relate to all applications known to the person skilled in the art in which some kind of sample(s)/mixture(s) need(s) to be processed at a certain temperature.

In particular, the present invention relates to means for covering one or more sample(s) that are suitable to avoid or minimize evaporation and/or condensation of any vaporizable substance that may be present in the sample(s) or reaction mixture(s), in particular evaporation of substance at the fringes of a vessel or an array of vessels or condensation of said substance on the lid of a reaction vessel or a plate/block containing the sample(s) and/or the means for covering.

### BRIEF DESCRIPTION OF THE DRAWINGS

FIG. 1 shows a preferred embodiment of the present invention in which the lid of the means for covering has a plane portion and an inclined portion. Upon closing the means for covering by sliding the means for covering over the means for accommodating (comprising the reaction vessel), the inclined portion of the contact area positions the plane portion of the contact area, wherein the overall contact area is moved with a vertical component relative to the plane of the reaction vessel.

FIG. 2 shows a preferred embodiment vis-à-vis the embodiment shown in FIG. 1, additionally comprising a circular belt facilitating the sliding of the inclined portion of the contact area by reducing the friction between the two.

FIG. 3 shows in a series of two panels another preferred embodiment according to the present invention in which the lid is lowered onto the horizontal plane defined by the reaction vessel in a continuous movement concurring simultaneously with a sliding movement of the means for covering closing said means for covering until the means come to their final position by hitting a stopping device. Therein, FIG. 3B shows the final operational state with the means for covering being in their final position and the lid being lowered into firm physical and thermal contact with the sample or reaction vessel.

### DETAILED DESCRIPTION OF THE INVENTION

The above described movement having a concurrent horizontal and vertical component can be performed manually and/or automatically (for example by means of an electronically controlled actuation) or by means of any combination of manual and automated action.

According to a preferred embodiment of the present invention, the means for positioning (24, 25) are realized as of a rail (24) that is preferably part of a base body (6) and/or of the means for accommodating (2) operated in conjunction with a corresponding movable rail member (25) that is preferably a part of or connected to the means for covering (3). Further preferably, the final position of the means for covering (3) relative to the means for accommodating (2) and/or the sample and/or reaction vessel(s) (1) is determined by means of the rail member (25) hitting the end of travel of the rail and/or the contact area—or a part thereof—hitting a stopping device (26) that preferably coincides with the end of travel of the rail (24). Preferably, said stopping device (26) is a part of the base body (6) and/or the means for accommodating (2).

In accordance with the present invention, no restrictions exist in regard to the specific realization of the rail (24) and the corresponding movable rail member (25) except for the requirement that the movable rail member (25) is part of the means for covering (3). A “rail” in accordance with the present invention is any device that allows and/or facilitates the movement of the means for covering (3) in one direction, preferably in the horizontal direction, so that the means for covering (3) preferably moves parallel to the plane as defined by the reaction vessel(s) (1) and/or the means for accommodating (2).

In a preferred embodiment, the rail (24) and the movable rail member (25) are adapted to enable a movement of the means for covering (3) relative to the means for accommodating (2) and/or the sample(s) of the reaction vessel(s) (1) that is essentially directed into one direction, preferably into the horizontal direction, i.e. so that the means for covering (3) move parallel in respect to the means for accommodating (2) and/or the base body (6) and/or the reaction vessel(s) (1).

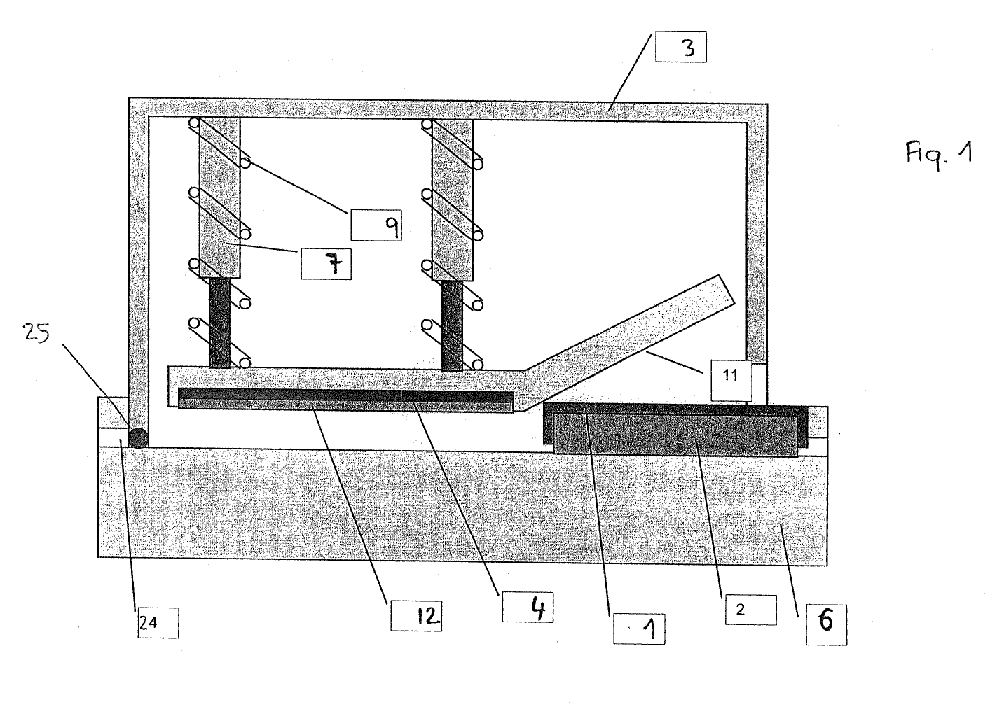

In one embodiment according to present invention (as shown, for example, in FIG. 1), the movement of the means for guiding (7), which preferably are connected to the means for covering (3) and further preferably are also connected to the lid (11, 12), is indirectly coupled with the movement of said means for covering (3).

In the context of the present invention, “indirect coupling” means that the means for guiding do not necessarily move in the vertical direction when the means for covering move in the horizontal direction. In the example shown in FIG. 1, said indirect coupling is provided by a sliding movement of the lid (11, 12), preferably mediated by an inclined portion of the lid (11). This sliding movement is preferably effected by the parallel movement of the means for covering (3) on the rail (24) while the lid (11, 12), in particular said inclined portion (11) of said lid is in direct physical contact with the sample and/or reaction vessel(s) (1) and/or the means for accommodating (2).

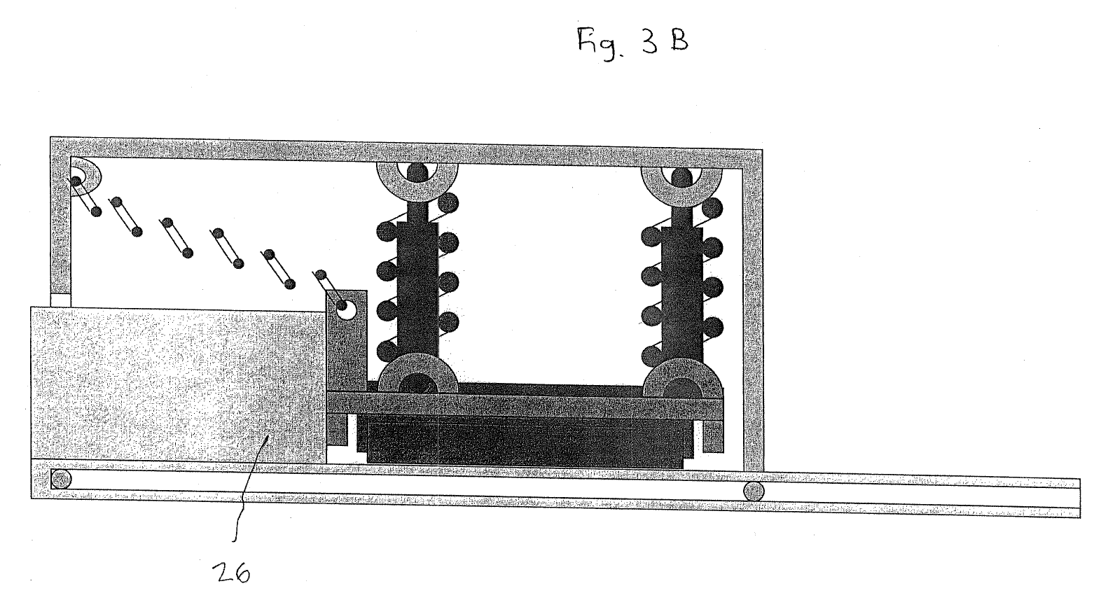

In another embodiment of the present invention as shown, for example, in FIG. 3, the movement of the means for guiding (7) is directly coupled to the movement of the means for covering (3), preferably by means of a spring (9′). In accordance with the present invention, “direct coupling” relates to any coupling that exerts a vertical force onto the means for guiding (7) whenever the means for covering (3) are moved in the parallel direction. In particular this embodiment, the means for guiding (7) may move, in particular with a component in the vertical direction, without the necessity of the contact area (12) being in physical contact with the reaction vessel(s) (1) and/or the means for accommodating (2).

No restrictions exist in regard to the specific realization of the means for guiding (7) as long as said means for guiding (7) allow for a movement of the lid (11, 12) which is not or is not entirely parallel to the horizontal plane, in particular not or not entirely parallel to the plane as defined by the sample and/or reaction vessel(s) (1) and/or the means for accommodating (2) and/or the base body (6).

In a preferred embodiment according to the present invention, said means for guiding (7) are realized as at least one, preferably as at least two, units capable of exerting a stroke movement. In a preferred embodiment, said unit(s) for stroke movement(s) is/are connected to both the means for covering (3) and the lid (11, 12).

It is preferred that said stroke movement is exerted by means of a spring or by means of a pneumatic movement or by any other conceivable piston-type=movement.

In a preferred embodiment, the pressure/force as exerted by the stroke movement of the means for guiding (7) can be preadjusted and/or controlled, for example by means of adjusting the spring force and/or by means of adjusting the travel of the rail member (25) on the rail (24). In a further preferred embodiment, the means for guiding (7) comprise as least one pressure exerting spring (9). More preferably, the pressure/force is predetermined such that it is sufficient to prevent the content of the vessel(s) from evaporating.

No restrictions exist in regard to the specific realization of the lid (11, 12). It is preferred that the lid (11, 12) is connected to the means for covering (3), preferably by means of the above-described means for guiding (7) that, in turn, are also preferably connected to the means for covering (3). Preferably the lid (11) comprises or is a contact area (12) that is preferably deformable.

In accordance with the present invention, the movement of the lid (11, 12) is realized so that it has both a horizontal and a vertical component in respect to the plane as defined by the base body (6) and/or the sample surface and/or the reaction vessel(s) (1) and/or the means for accommodating (2).

This is in contrast to the movement of the means for covering (3) as such, which preferably only has a parallel/horizontal component relative to the means for accommodating (2) and/or the base body (6) and/or the reaction vessel (1).

It is further preferred, that the lid (11, 12) comprises a material that is in direct contact with the reaction vessel (1) after alignment and/or closing of the means for covering (3), i.e. in the final operating position, wherein said material that is in direct contact with the reaction vessel(s) (1) is capable of withstanding the chemical and/or physical nature of the substances kept in the reaction vessel(s) (1) during reaction(s) and/or that is flexible enough to establish tight physical and/or thermal contact without damaging the reaction vessel(s) (1). For these purposes, a (disposable) sheet and/or a membrane and/or a pad is preferred which can be attached to and removed from the lid (11, 12), if necessary.

In one embodiment of the present invention as shown in FIG. 1, the lid (11, 12) preferably has an inclined portion (11) and a contact area (12) that is parallel to the plane as defined by the reaction vessel(s) (1) and/or the means for accommodating (2). It is preferred that the angle of the inclined portion relative to the horizontal plane is chosen so that the forces exerted onto the reaction vessel(s) upon sliding the contact area are not sufficient to damage the reaction vessel(s). It is preferred that said contact area and said inclined portion of the lid (11, 12) are integral to each other.

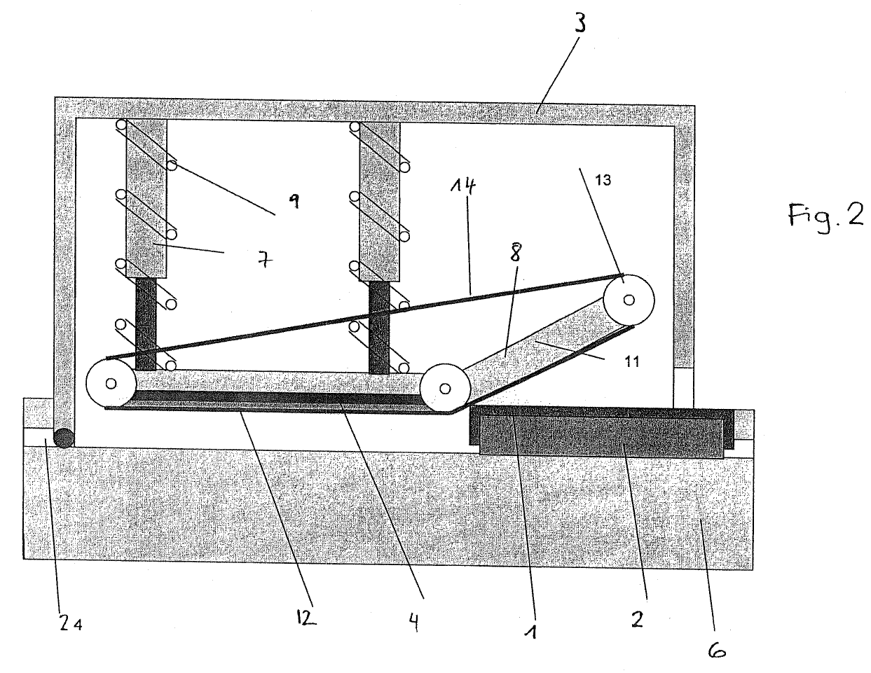

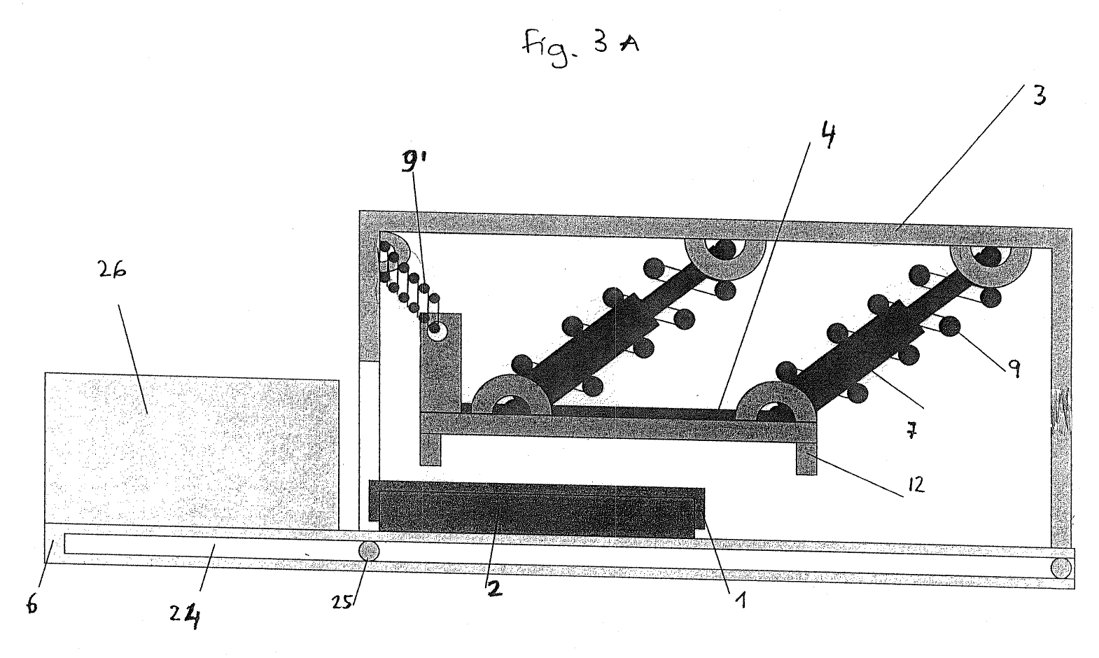

In a further embodiment in accordance with the present invention (as shown in FIG. 2) rolling means (14), preferably realized as a circular belt, are part of the lid (11, 12). In this embodiment, it is preferred that the circular movement of the circular belt is facilitated by means of guide rollers (13). This embodiment lowers or minimizes friction between the contact area and the reaction vessel(s).

In accordance with the present invention, no restrictions exist in regard to the at least one sample. The sample can be a single substance, a reaction mixture or any other conceivable material. Blind samples are included.

In a preferred embodiment, the at least one sample is contained in at least on reaction vessel and/or in at least one well/dimple/indentation of a plate, in particular a sample well plate (multititer plate, PCR plate) or a block, in particular a flat block. The sample may also be contained in a consumable/disposable that is placed on a flat block.

The reaction vessel, plate or block can be disposable or can be a permanent and/or integral part of the device, in particular of the means for accommodating.

No restrictions exist in regard to the reaction vessels that optionally contain the at least one sample. In fact, it is a particular advantage of the present invention that different types of reaction vessels may be used and that even different reaction vessels can be used within the same set of experiments and/or can be contained in one array of the same means for accommodating. In particular, the present invention allows for reaction vessels of different height and/or height tolerances to be used in combination with each other. In case a plurality of reaction vessels is arrayed in a plate or a block or any other type of reaction vessel holder, the present invention not only allows for sites in the plate or block or reaction vessel holder to be empty (i.e. to not contain a reaction vessel) but, in fact, provides means for covering that are particularly advantageous for such a setting.

The reaction vessels may be closed (i.e. may have a lid or cover or may be covered by a sheet or a film or foil) or may be open. According to the present invention, open reaction vessels can be used next to closed reaction vessels. Preferred reaction vessels are reaction tubes as known to the person skilled in the art as suitable for conducting PCR, including vessels having a flat bottom.

In a preferred embodiment, the reaction vessels, plates or blocks are sealed, for example, by means of caps (adding an additional height of approximately 1 to 2 mm to the overall height of the reaction vessel), in particular flat caps or domed caps or foils/films (having a thickness of approximately 0.02 mm).

No restrictions exist in regard to the means for heating and/or cooling. Preferably, the means are capable of heating or cooling at least one sample and/or at least one reaction vessel or plate or block. It is preferred that the means for heating and/or cooling are selected from the group of resistance heater, fluid mediated heating/cooling, air/gas cooling, Peltier heating/cooling, friction (Joule) heating/cooling, and/or radiation heating.

In a preferred embodiment according to the present invention, at least one means for heating and/or cooling at least one sample and/or reaction vessel is part of the means for covering. In this case, it is preferred that said means for heating and/or cooling minimizes or avoids evaporation of sample and/or minimizes or avoids condensation of vaporized sample on or in the vicinity of the means for covering.

It is further preferred, that an (additional) means for heating and/or cooling is provided in the means for accommodating a plurality of samples and/or reaction vessels.

In a further preferred embodiment, means for heating and/or cooling are provided as a part of the force distribution unit, in particular in conjunction with the medium or material that deforms continuously under the action of a shear force. In this context, it is particularly preferred that said medium or material is a fluid, in particular a gel or a liquid. It is further preferred, that the gel or the liquid has a high thermal conductivity (at least 0.1 W·m⁻¹·K⁻¹). It is further preferred that the liquid or the gel is in contact with a heat exchanger unit in order to change the temperature of the liquid/gel as it is brought in contact with the sample(s) and/or the reaction vessel(s). Preferably, such heat exchanger is a heating plug or a heating sheet.

No restrictions exist in regard to the means for accommodating at least one sample. This means may be a holder for reaction vessels or may be a block or a plate, for example a (flat) block made of metal, plastic materials or of composite materials that may comprise wells or dimples or any other type of indentation/containment.

The means for accommodating may be, for example, a (microtiter) plate, a water bath with an insert for holding reaction vessels, a carousel, any other type of multi-well plate or a flat block. Preferably, the means for accommodating are block- or box-shaped. It is preferred that said means are thermally insulated. It is further preferred that the means for accommodating comprise means for heating and/or cooling the reaction vessel(s) and/or the sample(s) from below and/or from the side.

The means for accommodating may be disposable or may be reusable. They may temporarily or permanently be part of a base body, or of any other part of the device according to the present invention.

No restrictions exist in regard to the means for covering at least one sample or at least one reaction vessel or the plate/block. The means for covering are preferably temporarily or permanently affixed to and/or aligned with the means for accommodating the sample(s) or reaction vessel(s). In this context, it is preferred that means for covering and the means for accommodating share a common base body. Further preferably, the unit comprising means for accommodating and the means for covering (optionally comprising a base body) completely enclose and/or encase the at least one sample or reaction vessel. Complete enclosing and/or encasing improves temperature stability.

In a preferred embodiment according to the present invention, the means for covering at least one sample or reaction vessel are in physical and thermal contact with at least one force distribution unit, wherein said force distribution unit is suitable to bring the at least one sample or reaction vessel in thermal contact with the means for covering and thereby establishes (direct) thermal contact between the force distribution unit and the sample(s) thus allowing efficient heating and/or cooling of the at least one sample. Tight mechanical and thermal contact between the sample and/or the reaction vessel, in particular the top thereof and/or the cover of the reaction vessel, on the one hand, and the force distribution unit (being part of the means for covering) on the other hand, is preferred for stable and efficient thermal processing of the chemical or biological process.

A preferred embodiment is shown in FIG. 1. A base body (6) comprises means for accommodating (2) supporting, in this case, one reaction vessel (1). The base body (6) also comprises a rail (24) on which the means for covering (3) can freely move in the horizontal direction [at least until a rail member (25) of the means for covering reaches the end of travel of the rail and/or hits a stopping device (not shown)].

The means for covering (3) of FIG. 1 comprise two parallel means for guiding (7) realized as holding posts being directed downward from the upper cover of the means for covering (3). A unit comprising a lid (11, 12) is suspended from said upper part of the means for covering (3) as mediated by said means for guiding (7). The vertical component of the movement of the lid (11, 12) relative to the reaction vessel (1) is achieved by means of pressure exerting springs (9) and a movable element of the means for guiding (7) capable of performing a stroke-movement in the downward direction.

The unit comprising a lid (11, 12) also comprises means for heating and/or cooling (4) to heat and/or cool the upper part of the reaction vessel (1). The contact area (12) establishes smooth contact between the reaction vessel (1) and the means for heating and/or cooling in the final (operating) position.

Upon moving the means for covering (3) from left to right, the inclined portion (11) of the contact area of the unit comprising the lid (11, 12) smoothly slides over reaction vessel (1) and brings the contact area (12) as pressured by springs (9) in tight and covering contact with the reaction vessel (1) while means for covering (3) reach their (final) closing position.

The embodiment shown in FIG. 2 essentially corresponds to the one shown in FIG. 1 with the additional feature of rolling means (14), here realized as a circular belt, facilitating the sliding of the inclined portion (12) of the lid onto the reaction vessel (1). The movement of the belt is facilitated by guide rollers (13).

The embodiment shown in FIG. 3 relates to another preferred embodiment in accordance with the present invention. Therein, the means for covering (3) comprise or are attached to a movable rail member (25) traveling in a rail (24) that is part of a base body (6). Said base body (6) supports means for accommodating (2) which in turn supports the reaction vessel (1). According to the embodiment shown in FIGS. 3A and 3B, the means for covering (3) are slid from the right to the left hand side until the rail member (25) reaches the end of travel of the rail (24) (see FIG. 3B).

The means for covering (3) comprise two means for guiding (7) that allow for a strike movement of the lid (12). In this specific embodiment, the movement of the lid (12) in both the vertical and the horizontal direction is achieved by a set of springs (9, 9′) coupling the downward movement of the means for guiding as attached to the contact area to the sliding movement of the means for covering (3) as illustrated by the sequence of movements shown in FIGS. 3A and 3B. Specifically, FIG. 3B shows the final position in which both the rail member (25) of the means for covering (3) has hit the end of travel of rail (24) and the unit comprising the lid (12) has engaged in the final position in respect to the reaction vessel (1) by hitting a stopping device (26). In this position, the lid (12) comprising means for heating and/or cooling (4) has been brought into tight physical and thermal contact with the reaction vessel (1) and a predetermined pressure is applied onto said reaction vessel (1) by means of pressure exerting springs (9) of the means for guiding (7).

All publications, patents and patent applications cited in this specification are herein expressly incorporated by reference to the same extent as if each individual publication, patent or application was specifically and individually indicated to be incorporated by reference.

The invention now being fully described, it will be apparent to one of ordinary skill in the art that many changes and modifications can be made thereto without departing from the spirit or scope of the appended claims.

### REFERENCE SIGNS

1 reaction vessel(s)
2 means for accommodating
3 means for covering
4 means for heating and/or cooling
6 base body
7 means for guiding
9 pressure exerting spring
9′ coupling spring
11 inclined portion of the lid
12 contact area of the lid
13 guide roller
14 rolling means
24 rail
25 movable rail member
26 stopping device

1. Device for controlling the temperature of at least one sample, wherein the device comprises at least the following components:
at least one means for accommodating (2) at least one sample;
at least one means for heating and/or cooling (4) at least one sample;
at least one means for covering (3) at least one reaction vessel, wherein the means for covering comprise at least one lid (11, 12);
at least one means for positioning (24, 25) the means for covering (3) relative to the at least one sample;
at least one means for guiding (7) the at least one lid (11, 12) of the means for covering (3) from a position of no thermal and/or physical contact with at least one sample into a position of thermal and/or physical contact with at least one sample,
characterized in that the means for positioning (24, 25) and the means for guiding (7) are adapted to effect a movement of the lid (11, 12) relative to sample, which within the same period of time, has a horizontal component and a vertical component.

2. Device according to claim 1, wherein the means for positioning (24, 25) and the means for guiding (7) are adapted to align the means for covering (3) relative to the sample and/or to the reaction vessel (1) and/or the means for accommodating (2) so that a contact area (12) of the lid is in physical and/or thermal contact with the at least one reaction vessel (1) and that the means for covering (3) and the lid (11, 12) are in a position in which the device is deemed closed and the thermal cycling/Processing can be started.

3. Device according to claim 1 or 2, wherein the contact area is connected to the means for covering (3) by means for guiding (7), wherein the means for guiding are preferably actuated by means of a spring force (9).

4. Device according to claim 3, wherein the contact area (12) additionally is directly connected to the means for covering (3) by means of a spring force (9′).

5. Device according to claim 3, wherein the lid (11, 12) comprises a contact area and an inclined portion.

6. Device according to any one of claims 1 to 5, wherein the means for guiding (7) are adapted to allow for a movement of the lid (11, 12) upon closing the means for covering that is not or is not entirely parallel to the horizontal plane, in particular to the plane as defined by the sample and/or by reaction vessel(s) (1) and/or the means for accommodating (2) and/or the base body (6), while the movement of the means for covering (3) as such preferably is entirely parallel to said horizontal plane.

7. Device according to claim 6, wherein said means for guiding (7) are realized as at least one, preferably as at least two, units capable of exerting a stroke movement.

8. Device according to any one of claims 1-4, wherein the lid (11, 12) comprises a circular belt.

9. Process for aligning and/or closing the cover of a device for controlling the temperature of at least one sample, wherein said process comprises at least the following steps:
(i) aligning and/or closing the means for covering (3) into a position in which the device is deemed closed, and
(ii) bringing at least one lid (11, 12) of at least one means for covering (3) from a position of no physical and/or thermal contact with at least one sample and/or reaction vessel into a position of physical and/or thermal contact with at least one sample and/or reaction vessel (1);
characterized in that at least these two process steps are performed simultaneously in one movement moving both the means for covering (3) and the lid (11, 12).

10. Process according to claim 9, wherein the movement of the means for covering (3) is parallel to the horizontal plane and the movement of the contact area, at the same time, has both a horizontal and a vertical component.

11. Process according to claim 10, wherein the movement having a concurrent horizontal and vertical component is effected during the process of closing the means for covering (3), in particular during the process of aligning the means for covering (3) relative to the sample and/or reaction vessel (1) and/or the means for accommodating (2) so that, at the conclusion of said movement, the means for covering (3) and the contact area (12) are in their predetermined final position.

12. Process according to any one of claims 9 to 11, wherein the means for covering (3) move in parallel relative to the means for accommodating (2) and/or the sample(s) and/or the reaction vessel(s) (1) wherein during said movement, the movement of the means for guiding (7) and the lid (11, 12) is occurring concurrently with the movement of the means for covering (3), wherein the movement of the lid preferably has a horizontal component, so that the lid and a vertical component, i.e. do not only move parallel to the means for accommodating (2) and/or the sample but also, to some extent, in the vertical direction, i.e. perpendicular to the horizontal plane as defined by the means for accommodating and/or the sample.

13. Use of the device according to any of claims 1 to 8 for carrying out chemical and/or biological reactions.

14. Use according to claim 13, wherein said chemical and/or biological reaction is the polymerase chain reaction.
